(19) United States
(12) Patent Application Publication
(10) Pub. No.: US 2003/0161875 A1
(43) Pub. Date: Aug. 28, 2003

(54) Fast dissolving tablets of cyclooxygenase-2 enzyme inhibitors

(76) Inventors: Deepak Murpani, New Delhi (IN); Vinod Kumar Arora, New Delhi (IN); Rajiv Malik, New Delhi (IN)

Correspondence Address: JAYADEEP R. DESHMUKH, RANBAXY PHARMACEUTICALS INC., 600 COLLEGE ROAD EAST, SUITE 2100, PRINCETON, NJ, 08540, US

(21) Appl. No.: 10/085,664

(22) Filed: Feb. 27, 2002

(51) Int. Cl. A61K 31/415; A61K 9/20

(52) U.S. Cl. 424/465; 514/406

(57) ABSTRACT

The present invention relates to fast dissolving tablets for oral administration comprising a therapeutically effective amount of drug(s) that acts selectively as a cyclooxygenase-2 (COX-2) enzyme inhibitor, which disintegrate quickly in mouth. The tablets are particularly suitable for patients who have difficulty in swallowing.

### FIELD OF THE INVENTION

The present invention relates to fast dissolving tablets for oral administration comprising a therapeutically effective amount of drug(s) that acts selectively as a cyclooxygenase-2 (COX-2) enzyme inhibitor, which disintegrate quickly in mouth. The tablets are particularly suitable for patients who have difficulty in swallowing.

### BACKGROUND OF THE INVENTION

Nonsteroidal anti-inflammatory drugs (NSAIDs) exert anti-inflammatory and analgesic effects through the inhibition of prostaglandin synthesis by blocking cyclooxygenase (COX) enzyme activity. COX enzyme has two isoforms: COX-1 and COX-2. COX-2 enzyme is inducible by inflammation, whereas COX-1 is present in most tissues as the house keeper enzyme. The inhibition of COX-1 is therefore undesirable whereas on the other hand inhibition of COX-2 enzyme accounts for the therapeutic benefits.

COX-2 inhibitors are the latest addition to the growing armamentarium of anti-inflammatory drugs. Much of the recent research has focussed upon efficacious methods for development of drug delivery of COX-2 enzyme inhibitors to treat inflammation associated maladies.

The ability of COX-2 inhibitors to selectively block formation of pro-inflammatory prostaglandins while sparing those that guard the gastrointestinal tract makes them an attractive choice for long term use, such as in rheumatoid arthritis or osteoarthritis.

Rheumatoid arthritis and osteoarthritis are old age diseases associated with joint pain, stiffness, inflammation or swelling. Many elderly persons have difficulty in taking conventional oral dosage forms like solutions, suspensions, tablets and capsules, because of hand tremors and dysphagia. Moreover, increase intake of water for swallowing conventional dosage forms results in frequent urination and nocturia.

Swallowing problems are also common in mentally ill, the developmentally disabled, and patients who are uncooperative, on reduced liquid-intake plans, or are nauseated. In cases such as motion sickness, sudden episodes of allergic attack or coughing, and an unavailability of water, swallowing the conventional tablets becomes difficult. Consequently, there is a need to provide a fast dissolving dosage form of COX-2 inhibitors for oral administration which disintegrates and dissolves rapidly in saliva without the need for drinking water.

### SUMMARY OF THE INVENTION

It is an object of the present invention to provide a fast dissolving tablet which comprises a therapeutically effective amount of drug(s) that acts as a cyclooxygenase-2 enzyme (COX-2) inhibitor for oral administration which disintegrate quickly in the mouth. The tablets prepared by the present invention disintegrate and dissolve in the oral cavity in less than about 30 seconds without the need of water. The fast dissolving tablet of COX-2 of the present invention process has pleasant mouth feel and there is no after taste or grittiness.

The fast dissolving tablets according to the present inventions comprises a therapeutically effective amount of COX-2 inhibitor, a filler, and optionally other pharmaceutical excipients.

Direct compression is the preferred method because of the above advantages. Accordingly, the present invention provides a process for the preparation of fast dissolving tablets comprising a therapeutically effective amount of drug(s) that acts as a cyclooxygenase-2 (COX-2) inhibitor for oral administration.

The fast dissolving tablets of the present invention can either be produced by conventional methods like wet granulation, dry granulation and direct compression or by specialized techniques like tablet molding and freeze drying.

Since the pharmaceutical industry is constantly making efforts to increase the efficiency of tabletting operations and reduce costs by utilizing the smallest amount of floor space and labor as possible for a given operation, increasing attention is being given to direct compression of tablet preparation.

Direct compression is regarded as a relatively quick process where the powdered materials are compressed directly without changing the physical and chemical properties of the drug. The advantages of direct compression include

(i) few manufacturing steps, as granulation step is eliminated

(ii) elimination of heat and moisture and therefore better

a. physical stability such as no change in crystallinity and polymorphic form of the drug, and

b. chemical stability

(iii) use of conventional equipment and commonly available excipients; and

(iv) low cost and less manpower.

The process comprises:

a) blending a therapeutically effective amount of COX-2 inhibitors with filler and optionally, other pharmaceutical acceptable excipients, for a time sufficient to form a homogeneous mixture.

b) compressing the homogeneous mixture obtained in step (a) to form the fast dissolving tablet of COX-2 inhibitor.

### DETAILED DESCRIPTION OF THE INVENTION

According to the present invention, the “COX-2 inhibitor” as used herein to embrace compounds that specifically/selectively, or preferentially inhibits cyclooxygenase-2 over cyclooxygenase-1. Illustrative examples of COX-2 enzyme inhibitors that are advantageously administered by the pharmaceutical compositions of this invention include “specific inhibitors” such as celecoxib, rofecoxib, parecoxib, valdecoxib, and the like or “preferential inhibitors” such as meloxicam, nimesulide, etodolac, nabumetone, and the like.

“Therapeutically effective amount” means that amount of a drug or pharmaceutical agent that will elicit the biological or medical response of a tissue, a system, animal or human that is being sought by a researcher, veterinarian, medical doctor or other clinician.

Fillers of the present invention can be selected from any such pharmaceutically acceptable excipient, which gives bulk to the COX-2 inhibitor composition and which is physically and chemically compatible with COX-2 inhibitor; preferably those fillers may be selected from alkali earth metal salts such as directly compressible dicalcium phosphate dihydrate, tricalcium phosphate, calcium sulfate, calcium carbonate, calcium hydroxide, aluminium hydroxide, magnesium silicate, aluminium magnesium hydroxide; carbohydrates such as directly compressible maltose, maltitol, sorbitol, mannitol, glucose, sucrose, xylitol, lactose, lactose monohydrate, erythritol, fructose, maltodextrins; celluloses such as microcrystalline cellulose, calcium carboxy methyl cellulose; starches such as pregelatinized starch, potato starch, maize starch; clays such as kaolin and polyethylene glycols (PEG) such as PEG 4000; or mixtures thereof.

The effective amount of the fillers found useful in the present invention is in the range of about 10 to about 95 weight percent, preferably about 25 to about 85 weight percent, and most preferably about 80 weight percent of the COX-2 inhibitor compositions of this invention. One of the preferred fillers is directly compressible mannitol.

The direct compression excipients are chosen such that they have good flow and compressible characteristics and prevent segregation of powders in the hopper and thereby help in direct compression.

The optional pharmaceutical excipients of this invention may be selected from the binders, disintegrants, lubricants, glidants, colouring agents, flavouring agents and sweeteners which are chemically and physically compatible with COX-2 enzyme inhibitors.

The direct compression method for preparing tablets requires a material that not only is free-flowing but also sufficiently cohesive to act as a binder. Materials such as microcrystalline cellulose, microcrystalline dextrose, mannitol, directly compressible dicalcium phosphate, amylose and polyvinylpyrrolidone have such properties.

Disintegrants preferred for the present invention may be selected from starches or modified starches such as sodium starch glycolate, corn starch, potato starch or pregelatinized starch; clays such as bentonite, montmorillonite or veegum; celluloses such as microcrystalline cellulose, hydroxypropyl cellulose or carboxymethyl cellulose; algins such as sodium alginate or alginic acid; cross-linked cellulose such as croscarmellose sodium; gums such as guar gum or xanthan gum; cross-linked polymers such as crospovidone; effervescent agent such as sodium bicarbonate and citric acid; or mixtures thereof.

The effective amount of a disintegrant found useful for the COX-2 inhibitor compositions of this invention is in the range of about 1.0 to about 10.0 weight percent, preferably about 1.5 to about 2.5 weight percent and most preferably about 2.0 weight percent of the COX-2 inhibitor compositions by this invention. The preferred disintegrant is croscarmellose sodium.

The lubricants of the present invention may be selected from talc, magnesium stearate, calcium stearate, stearic acid, magnesium lauryl sulphate and hydrogenated vegetable oil. Soluble lubricants include sodium benzoate, a mixture of sodium benzoate and sodium acetate, sodium chloride, leucine, sodium stearyl fumarate and PEG 4000.

The effective amount of lubricant found useful in the present invention is in the range of about 0.25 to about 4 weight percent, preferably about 0.5 to about 2 weight percent, and most preferably 1.0 weight percent of the COX-2 inhibitor compositions of this invention. The preferred lubricant is magnesium stearate.

The glidants of the present invention may be selected from colloidal silicon dioxide and talc.

The coloring agent of the present invention may be selected from any colorant used in pharmaceuticals which is approved and certified by the FDA.

The flavouring agent of the present invention include both natural and artificial flavours such as artificial vanilla, cinnamon, various fruit flavours, both individual and mixed; mints such as peppermint, menthol; essential oils such as thymol, eculyptol and methyl salicylate and the like. The flavours are generally utilized in amounts that will vary depending upon the individual flavour, and may range in amounts of about 0.5% to about 3% by weight of the final composition weight.

The sweeteners for the present invention include both natural and artificial sweetners. The sweetners may include, among others, water-soluble sweetening agents such as monosaccharides, disaccharides and polysaccharides such as xylose, ribose, glucose, mannose, galactose, fructose, dextrose, sucrose, maltose, partially hydrolyzed starch, or corn syrup solids and sugar alcohols such as sorbitol, xylitol, mannitol and mixtures thereof; water-soluble artificial sweeteners such as the soluble saccharin salts, cyclamate salts, acesulfam-K and the like, and free acid form of saccharin and dipeptide based sweeteners. The amount of sweetener will vary with the desired sweeteners selected for a particular tablet composition.

The process of the present invention comprises sieving of the COX-2 inhibitors, fillers, disintegrants, binders, glidants, colouring agents, flavouring agents and sweeteners, through a suitable sieve and admixing them to make a uniform blend. The lubricant is also passed through the suitable sieve and mixed with the blend. The blend is directly compressed using the suitable tooling.

The tablets made by the present inventive process disintegrate/dissolve in less than about 30 seconds preferably in about 25 seconds. The process of this invention for preparing rapidly dissolving tablet may be used for any strength of COX-2 inhibitor tablets without deviating from this invention.

The present invention is illustrated by, but by no means limited to, the following examples:

### EXAMPLE 1

Rofecoxib mouth dissolving tablets-25 mg.

| Ingredient | Quantity (mg) |
|---|---|
| Rofecoxib | 25.28 |
| Aspartame | 0.35 |
| Mannitol | 166.67 |
| Croscarmellose sodium | 4.00 |
| Colloidal silicon dioxide | 1.00 |
| Mixed fruit flavour | 0.70 |
| Magnesium stearate | 2.00 |
| Total | 200.00 |

1. Rofecoxib, aspartame, mannitol, croscarmellose sodium, colloidal silicon dioxide and mixed fruit flavour are sifted through the sieve #44 BSS and admixed for about 15 minutes to make a uniform blend.

2. Magnesium stearate is passed through sieve #100 BSS and mixed with the blend of step 1 for sufficient time.

3. Uniform blend of step 2 is directly compressed using 9 mm, round biconcave tooling to make the tablets of about 3.8±0.1 mm thickness.

The mouth dissolving tablets prepared by the above composition and process had hardness in the range of 2.2 to about 4.0 Kp. The disintegration time in water was less than 15 seconds, whereas the mouth dissolving time was less than 25 seconds. The friability was about 0.4% w/w. The mouth dissolving rofecoxib tablets are tested in 1% sodium lauryl sulphate (SLS) according to the procedure described in the United States Pharmacopoeia XXIII, Apparatus 1 @ 100 rpm and found to have the following release profile:

| Time (Minutes) | % Rofecoxib dissolved |
|---|---|
| 15 | 74 |
| 30 | 83 |
| 45 | 88 |

### EXAMPLE 2

| Ingredient | Quantity (mg) |
|---|---|
| Rofecoxib | 50.56 |
| Aspartame | 0.70 |
| Mannitol | 333.34 |
| Croscarmellose sodium | 8.0 |
| Colloidal silicon dioxide | 2.0 |
| Mixed fruit flavour | 1.4 |
| Magnesium stearate | 4.0 |
| Total | 400.0 |

The procedure of Example 1 was followed to prepare the tablets of above composition.

The rofecoxib mouth dissolving tablet of 50 mg strength had an average weight of 400±20 mg, thickness of 4.9±0.2 mm, hardness of 4.5-5.0 Kp, disintegration time of less than 20 seconds, mouth dissolving time of about 25 seconds, friability of about 0.44% w/w and dissolution upto 86% in 45 minutes.

### EXAMPLE 3

Nimesulide mouth dissolving tablet-100 mg.

| Ingredient | Quantity (mg) |
|---|---|
| Nimesulide | 100.00 |
| Aspartame | 4.5 |
| Mannitol | 318.75 |
| Croscarmellose sodium | 10.5 |
| Colloidal silicon dioxide | 2.25 |
| Orange flavour | 4.5 |
| Monosodium citrate | 5.0 |
| Magnesium stearate | 4.5 |
| Total | 450.0 |

The procedure of Example 1 was followed to prepare the above tablets.

The nimesulide mouth dissolving tablet of 100 mg strength had an average weight of 450±22.5 mg, thickness of 5.7±0.2 mm, hardness of 2-5 Kp, disintegration time of less than 20 seconds, mouth dissolving time of about 25 seconds and friability of about 0.9% w/w.

While the present invention has been described in terms of its specific embodiments, certain modifications and equivalents will be apparent to those skilled in the art and are intended to be included within the scope of the present invention.

We claim:

1. A fast dissolving tablet which comprises a therapeutically effective amount of drug(s) that acts as a cyclooxygenase-2 (COX-2) inhibitor for oral administration.

2. The tablet according to claim 1 wherein the tablet comprises a therapeutically effective amount of COX-2 inhibitor, a filler and optionally, other pharmaceutical excipients.

3. The tablet according to claim 1 wherein the fast dissolving tablet dissolves in the mouth.

4. The tablet according to claim 1 or 2 wherein the drug(s) that acts as a cyclooxygenase-2 (COX-2) inhibitor is specific or preferential COX-2 inhibitor.

5. The tablet according to claim 4 wherein the COX-2 inhibitor is selected from the group consisting of meloxicam, rofecoxib, celecoxib, valdecoxib, parecoxib, nabumetone, nimesulide and etodolac.

6. The tablet according to claim 2 wherein the filler may be selected from the group consisting of alkali earth metal salts, carbohydrates, celluloses, starches, clays and polyethylene glycols, and mixtures thereof.

7. The tablet according to claim 9 wherein the filler may be selected from the group consisting of directly compressible dicalcium phosphate dihydrate, tricalcium phosphate, calcium sulfate, calcium carbonate, calcium hydroxide, aluminium hydroxide, magnesium silicate, aluminium magnesium hydroxide, maltose, maltitol, sorbitol, mannitol, glucose, sucrose, xylitol, lactose, lactose monohydrate, erythritol, fructose, maltodextrins, microcrystalline cellulose, calcium carboxy methyl cellulose, pregelatinized starch, potato starch, maize starch, kaolin, polyethylene glycol 4000, and mixtures thereof.

8. The tablet according to claim 2 wherein the pharmaceutical excipients comprises binders, disintegrants, lubricants, glidants, colouring agents, flavouring agents and sweeteners.

9. The tablet according to claim 8 wherein the binders may be selected from the group consisting of microcrystalline cellulose, mannitol, microcrystalline dextrose, directly compressible dicalcium phosphate, amylose and polyvinylpyrrolidone.

10. The tablet according to claim 8 wherein the disintegrant is selected from the group consisting of starches or modified starches, clays, celluloses, algins, cross-linked celluloses, gums, cross-linked polymers, effervescent agents, and mixtures thereof.

11. The tablet according to claim 10 wherein the disintegrant is selected from the group consisting of sodium starch glycolate, corn starch, potato starch, pregelatinized starch, bentonite, montmorillonite, veegum, microcrystalline cellulose, hydroxypropyl cellulose, carboxymethyl cellulose, sodium alginate, alginic acid, croscarmellose sodium, guar gum, xanthan gum, crospovidone; sodium bicarbonate and citric acid, and mixtures thereof.

12. The tablet according to claim 8 wherein the lubricants may be selected from the group consisting of talc, magnesium stearate, calcium stearate, stearic acid, magnesium lauryl sulphate and hydrogenated vegetable oil, sodium benzoate, sodium acetate, sodium chloride, leucine, sodium stearyl fumarate, PEG 4000, and mixtures thereof.

13. The tablet according to claim 8 wherein the glidants may be selected from the group consisting of colloidal silicon dioxide and talc.

14. The tablet according to claim 8 wherein the colouring agents may be selected from any colorant used in pharmaceuticals which is approved and certified by the FDA.

15. The tablet according to claim 8 wherein the flavouring agent may be selected from the group consisting of natural and artificial flavours, mints and essential oils or the mixtures thereof.

16. The tablet according to claim 15 wherein the flavouring agent may be selected from the group consisting of peppermint, menthol, artificial vanilla, cinnamon, various fruit flavours, both individual and mixed, thymol, eculyptol and methyl salicylate and the like.

17. The tablet according to the claim 8 wherein the sweetener may be selected from the group consisting of natural and artificial sweeteners.

18. The tablet according to the claim 17 wherein the sweetener may be selected from the group consisting of monosaccharides, disaccharides, polysaccharides, partially hydrolyzed starch, corn syrup solids, sugar alcohols, water-soluble artificial sweeteners, and mixtures thereof.

19. The tablet according to the claim 18 wherein the sweetener may be selected from the group consisting of xylose, ribose, glucose, mannose, galactose, fructose, dextrose, sucrose, maltose, sorbitol, xylitol, mannitol, soluble saccharin salts, cyclamate salts, acesulfam-K and free acid form of saccharin and dipeptide based sweeteners, and mixtures thereof.

20. A mouth dissolving tablet of COX-2 inhibitor consisting of a COX-2 inhibitor, croscarmellose sodium, mannitol, aspartame, colloidal silicon dioxide, magnesium stearate and flavouring agent.

21. A process for preparing a fast dissolving tablet according to claim 2 comprising the steps of:
(a) blending a therapeutically effective amount of COX-2 inhibitor, a filler, and optionally, other pharmaceutical excipients;
(b) compressing the homogeneous mixture obtained in step (a).

22. The process according to claim 21 wherein the blend is granulated before compression.

23. The process according to claim 22 wherein the granulation is done by wet or dry granulation methods.

24. The process according to claim 23 wherein the dry granulation is done by slugging or roller compaction.
